(19) United States
(12) Patent Application Publication
(10) Pub. No.: US 2003/0040646 A1
(43) Pub. Date: Feb. 27, 2003

(54) Process for the hydroxylation of aromatic hydrocarbons

(76) Inventors: Uwe Tanger, Bochum (DE); Johan Van Den Tillaart, Broekhuizenvorst (NL)

Correspondence Address: KNOBBE MARTENS OLSON & BEAR LLP, 2040 MAIN STREET, FOURTEENTH FLOOR, IRVINE, CA, 92614, US

(21) Appl. No.: 10/190,748

(22) Filed: Jul. 5, 2002

(30) Foreign Application Priority Data
Jul. 7, 2001 (DE) 101 33 067.7

(51) Int. Cl. C07C 37/58

(52) U.S. Cl. 568/800

(57) ABSTRACT

The present invention relates to industrial applications of nitrous oxide, e.g. the hydroxylation of benzene to phenol, requires large quantities of nitrous oxide having a very low impurity content. According to the present invention, nitrous oxide which is obtained by reaction of ammonia with nitric acid can be used for the hydroxylation of aromatics, the use of nitrous oxide from the reaction of ammonia with nitric acid in particular being economically advantageous because all starting materials are available in sufficient quantity and at a favorable price.

### FIELD OF THE INVENTION

The invention relates to a process for the hydroxylation of aromatic hydrocarbons by means of nitrous oxide, in which the nitrous oxide is obtained by reaction of ammonia with nitric acid.

### DESCRIPTION OF THE RELATED ART

An important aromatic hydroxy compound is phenol which is used, in particular, for preparing bisphenol A and its downstream products. Phenol is nowadays mainly prepared by the Hock process from cumene, with acetone being produced as coproduct. The economics of this process are dependent, in particular, on the market for the coproduct acetone. Phenol production without formation of the coproduct acetone could circumvent this difficulty.

In terms of a coproduct-free synthesis of phenol, the direct oxidation of benzene by means of dinitrogen oxide (nitrous oxide) in the gas phase over pentasils or catalysts based on vanadium pentoxide on silica or zeolites is, owing to its high selectivity (up to 98%) at a comparatively high conversion (from 10 to 30%), one of the most promising processes and is described, for example, in WO 95/27560. The catalyzed direct hydroxylation of benzene by means of nitrous oxide to form phenol was described for the first time by Iwamoto et al., J. Phys. Chem., 87(6): 903, 1983. The direct synthesis of phenol from benzene and nitrous oxide makes it possible in principle to achieve an industrial preparation of phenol which is decoupled from acetone and led only by the world market for phenol. Such a process for preparing phenol is described, for example, by WO 95/27691.

A problem which arises in the operation of such a plant for the direct oxidation of benzene is the provision of a sufficient amount of nitrous oxide. A production capacity of such a plant for the direct oxidation of benzene of 400,000 metric tons/year of phenol requires 190,000 metric tons/year of nitrous oxide. The nitrous oxide should be very pure, since traces of NOₓ or NH₃ poison the catalysts which are preferably used in the oxidation of the benzene.

WO 00/01654 describes the provision of nitrous oxide for the direct oxidation of benzene by use of offgas from the production of adipic acid. However, the amount of nitrous oxide formed in the preparation of adipic acid is far short of what is required to cover a significant part of the demand for phenol from direct oxidation of benzene. In addition, the nitrous oxide obtained as offgas from adipic acid production contains appreciable amounts of nitrogen and small amounts of NOₓ.

EP 0 799 792 describes the provision of nitrous oxide by oxidation of NH₃. This reaction of NH₃ with atmospheric oxygen gives a nitrous oxide which has a high nitrogen content. Nitrogen and nitrous oxide can be separated from one another by liquefaction of the nitrous oxide, but this separation requires a great deal of energy. If a large amount of nitrogen remains in the nitrous oxide because the removal of the nitrogen is too energy-intensive, some apparatuses in the plant for the direct oxidation of benzene have to have appropriately large dimensions. These large dimensions result in increased capital and operating costs which significantly worsen the economics of a direct oxidation process compared to conventional processes for the preparation of phenol, e.g. by the Hock process. A high proportion of nitrogen in the nitrous oxide also makes the work-up of the offgas stream more difficult. The offgas stream usually contains unreacted benzene which has to be removed from the offgas stream for economic reasons and to comply with emission laws. The large amount of ballast nitrogen, i.e. nitrogen which has not participated in the actual reaction, makes the quantities of offgas stream to be worked up unnecessarily larger and thus makes the overall process uneconomic.

In the laboratory and also on the industrial scale, the preparation of nitrous oxide is frequently carried out by thermal decomposition of ammonium nitrate, cf. Römpp Chemie Lexikon, 9th Edition, Vol. 5, 1992, keyword “nitrogen oxides”. Such a process is described in, for example, U.S. Pat. No. 4,154,806. However, the thermal decomposition of ammonium nitrate is strongly exothermic and can occur as a detonation at elevated temperature. For this reason, the decomposition of ammonium nitrate is carried out in a melt of ammonium hydrogen sulfate and ammonium sulfate. The reaction is quite difficult to control, as evidenced by U.S. Pat. No. 4,154,806. In addition, ammonium nitrate itself is a relatively valuable starting material, since the preparation of ammonium nitrate as solid is relatively costly.

U.S. Pat. No. 4,102,986 likewise describes a process for preparing nitrous oxide from ammonium nitrate. In this process, chloride ions are used as catalyst. Such reaction mixtures in which chloride ions are present are highly corrosive and therefore frequently require the use of special materials, e.g. titanium- or tantalum-plated steel, which are therefore expensive, as materials of construction for the reactor.

In some uses of nitrous oxide, the presence of other nitrogen oxides has to be avoided as far as possible. Thus, for example, in-house studies on the hydroxylation of benzene by means of nitrous oxide to form phenol have shown that even small traces of nitrogen monoxide in the nitrous oxide can cause the catalytic phenol synthesis from benzene and nitrous oxide to stop.

U.S. Pat. No. 3,656,899 describes the preparation of nitrous oxide from ammonium nitrate which is obtained by reaction of ammonia with nitric acid. However, no indication is given that this nitrous oxide would be suitable for the direct oxidation of benzene to phenol.

DE 100 09 639 describes a process for preparing nitrous oxide which is suitable for use in the hydroxylation of aromatics. In this process, nitrous oxide is prepared by reduction of nitrogen monoxide by means of a reducing agent. However, this process for preparing nitrous oxide is relatively expensive.

### SUMMARY OF THE INVENTION

It is therefore an object of the present invention to provide a simple and economical process for the hydroxylation of aromatics by means of nitrous oxide, which not only has a high potential for industrial use but also gives a high yield of hydroxylated aromatics on the basis of conventional catalysts with very little by-product formation.

### BRIEF DESCRIPTION OF THE DRAWINGS

The process of the invention is explained in more detail with the aid of FIG. 1 and FIG. 2, without the process being restricted thereto. FIG. 1 shows the concentration as a function of time in a hydroxylation of benzene according to the invention as described in Example 1.

FIG. 2 schematically shows the process of the invention. Ammonia and air are fed into the nitric acid plant S. In this plant, nitric acid, nitrogen and water are produced from these starting substances. The nitric acid is fed together with further ammonia into the nitrous oxide plant L in which the starting substances are converted into nitrous oxide and water. The nitrous oxide formed by the reaction of nitric acid and ammonia is transferred to a benzene hydroxylation plant B into which benzene is also fed. At the outlet of the plant B, the product phenol and nitrogen are obtained.

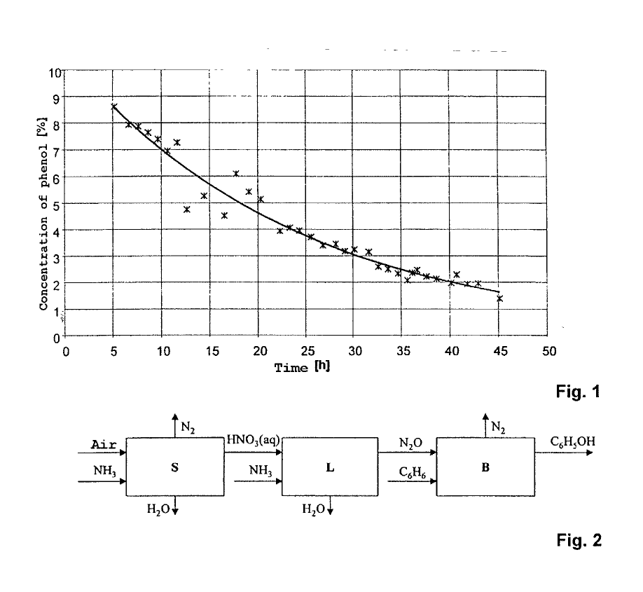

### DETAILED DESCRIPTION OF THE PREFERRED EMBODIMENT

It has surprisingly been found that nitrous oxide obtained by reaction of ammonia with nitric acid is very suitable for direct use in the hydroxylation of aromatics, since it has a low proportion of other nitrogen oxides and a significantly lower nitrogen content than nitrous oxide prepared by conventional methods.

The present invention accordingly provides a process for the hydroxylation of aromatics by means of nitrous oxide, in which nitrous oxide prepared by reaction of ammonia with nitric acid is used.

The process of the invention has the advantage that the nitrous oxide obtained by reaction of ammonia and nitric acid can, as relatively pure nitrous oxide, be used directly for the oxidation of benzene to phenol. The nitrous oxide obtained by this reaction is usually relatively pure, and any slight contamination by traces of other nitrogen oxides (NOx) can be removed in a simple manner by scrubbing.

The process of the invention in which nitrous oxide obtained by reaction of ammonia with nitric acid is used for the oxidation of aromatics makes it possible to use plants for direct oxidation which are smaller than plants of this type through which a high nitrogen ballast has to be conveyed together with the nitrous oxide. As a result of the reduced mass flows, the process of the invention enables the capital and production costs to be significantly reduced compared to direct oxidation by conventional processes.

As a result of the low nitrogen content of the nitrous oxide used, the product gas stream from the hydroxylation also contains smaller amounts of nitrogen than in the case of conventional processes, so that the removal of the nitrogen from the products is simplified.

For the purposes of the present invention, the term “other nitrogen oxides” refers to all nitrogen-oxygen compounds with the exception of nitrous oxide (N₂O), in particular the compounds NO, NO₂ and N₂O₄.

The process of the invention is described by way of example below, without being restricted thereto.

In the process of the invention for the hydroxylation of aromatics, nitrous oxide obtained by reaction of ammonia with nitric acid is used as oxidant.

The preparation of nitrous oxide is preferably carried out as described in U.S. Pat. No. 3,656,899 by reacting ammonia with nitric acid; details of the way in which the reaction is carried out can be taken from this document. The reaction is preferably carried out in the liquid phase, with an ammonia atmosphere being present above the liquid phase. The reaction of ammonia with nitric acid forms ammonium nitrate which is decomposed into nitrous oxide and water. The ammonium nitrate is usually present in aqueous solution in the liquid phase.

The reaction is preferably started by placing nitric acid, preferably concentrated 60% strength by weight nitric acid, in a reaction vessel. Ammonia is passed into this reaction vessel. The ammonia can be passed in gaseous form through the nitric acid. It can likewise be advantageous to introduce an aqueous solution of ammonia into the nitric acid; thorough mixing of the ammonia-containing phase with the nitric acid has to be ensured both when the ammonia is introduced as liquid phase and when it is introduced as gaseous phase. This can be achieved, for example, by means of static mixers or other apparatuses customarily used for mixing liquid/liquid or liquid/gas mixtures. The reaction of ammonia with nitric acid forms ammonium nitrate which reacts further in the aqueous solution to form water and nitrous oxide. Both the formation of ammonium nitrate and the decomposition of the ammonium nitrate into nitrous oxide and water are exothermic reactions. It is naturally also possible to use commercially available ammonium nitrate directly, but this is not entirely safe to handle and, in addition, is relatively expensive compared to ammonia and nitric acid.

The abovementioned reactions are advantageously carried out at a temperature of from about 80 to about 200° C., preferably from about 80 to about 105° C., from about 100 to about 160° C. or from about 165 to about 200° C. and very particularly preferably at a temperature of from about 100 to about 140° C. The temperature of the liquid phase in the reaction vessel is preferably set to a temperature in the range from about 80 to about 200° C. This can be achieved, for example, by providing an apparatus for the removal of heat of reaction in the reaction vessel. This apparatus is preferably a heat exchanger which can advantageously be connected in such a way that it cannot only remove heat of reaction from the reaction vessel but can also introduce heat energy into the liquid phase in the reaction vessel when needed, e.g. when the reaction is started.

The liquid phase in the reaction vessel preferably comprises from about 15 to about 35% by weight, more preferably from about 15 to about 21% by weight, from about 21 to about 28% by weight or from about 28 to about 35% by weight, of nitric acid, from about 30 to about 50% by weight, more preferably from about 30 to about 40% by weight or from about 40 to about 50% by weight, of ammonium nitrate, from about 15 to about 55% by weight, more preferably from about 15 to about 45% by weight, from about 20 to about 50% by weight or from about 25 to about 55% by weight, of water and from about 0.01 to about 0.2% by weight, more preferably from about 0.02 to about 0.1% by weight, of chloride ions.

It can be advantageous for the liquid phase in the reaction vessel to comprise at least one catalyst which catalyzes, i.e. speeds or assists, the reaction of ammonia with nitric acid or the decomposition of ammonium nitrate into water and nitrous oxide. A catalyst which promotes the decomposition of ammonium nitrate is preferably present in the liquid phase. Suitable catalysts are, in particular, those which comprise manganese, copper, cerium, lead, bismuth, cobalt or nickel or mixtures of these metals. The catalysts can be present as homogeneous catalysts, e.g. in the form of metal salts such as lead nitrate, copper nitrate, copper chloride, nickel chloride or cobalt chloride. However, it can likewise be advantageous for the catalysts to be used according to the invention to be present as heterogeneous catalysts, e.g. as zeolitic or silicalitic compounds containing manganese, copper, cerium, lead, bismuth, cobalt or nickel or mixtures of these metals.

The molar ratio of metal catalyst to ammonium nitrate in the reaction vessel is preferably from about 1:1 000 000 to about 1:1 000, preferably from about 1:1 000 000 to about 1:10 000.

In the reaction according to the invention of ammonia with nitric acid to form ammonium nitrate, the latter decomposes into nitrous oxide and water. The nitrous oxide rises from the liquid phase and mixes with the ammonia atmosphere above the liquid phase. The nitrous oxide can be extracted from the ammonia atmosphere in various ways. One possible method of extraction is, for example, to pass the ammonia atmosphere comprising nitrous oxide through a column through which a liquid phase comprising nitric acid is passed in countercurrent. The ammonia combines with the nitric acid to form ammonium nitrate and the liquid phase in which the ammonium nitrate is formed can be recirculated to the reaction vessel. A detailed description of the process may be found, for example, in U.S. Pat. No. 3,656,899. At the top of the column, crude nitrous oxide containing less than about 15% by weight, preferably less than about 10% by weight and very particularly preferably less than about 3% by weight, of impurities can be taken off. The crude nitrous oxide preferably contains less than about 10% by weight of nitrogen, particularly preferably less than about 5% by weight of nitrogen and very particularly preferably less than about 2% by weight of nitrogen. The crude nitrous oxide also preferably has a content of other nitrogen oxides of less than about 1% by weight, more preferably less than about 0.1% by weight, and particularly preferably has a proportion of other nitrogen oxides of less than about 50 volume parts per million (vppm), preferably less than about 20 vppm and very particularly preferably less than about 10 vppm.

The reaction occurring in the reaction according to the invention of ammonia with nitric acid to give nitrous oxide is:

NH₃ +HNO₃→NH₄NO₃→N₂O+6H₂O+1/2O₂ (I)

Depending on the impurities present in the crude nitrous oxide, this can be used directly for the hydroxylation of aromatics or can firstly be freed of these impurities. Impurities which can be present in the crude nitrous oxide include, inter alia, other nitrogen oxides, water and nitrogen. The nitrogen can be removed from the nitrous oxide by, for example, condensation of the nitrous oxide. The water can be removed from the nitrous oxide by freezing out or by means of a suitable desiccant. The other nitrogen oxides can be separated from the nitrous oxide by scrubbing by means of a mixture of an oxidant, e.g. potassium permanganate or hydrogen peroxide, and an aqueous alkali, e.g. aqueous sodium or potassium hydroxide. It can be advantageous for the crude nitrous oxide firstly to be freed of other nitrogen oxides by scrubbing, subsequently be freed of water in an appropriate manner and finally be freed of nitrogen by condensation. If the crude nitrous oxide has a content of other nitrogen oxides of less than about 50 vppm, preferably less than about 20 vppm and very particularly preferably less than about 10 vppm, scrubbing can be omitted.

According to the invention, nitrous oxide which has been prepared by reaction of ammonia with nitric acid is used for the hydroxylation of aromatics and preferably has a nitrogen content of less than about 5% by weight, preferably less than about 2% by weight. It has surprisingly been found that crude nitrous oxide obtained by reaction of ammonia and nitric acid is particularly suitable for hydroxylation when it has a content of other nitrogen oxides of less than about 50 volume parts per million (vppm), preferably less than about 20 vppm and very particularly preferably less than about 10 vppm.

The above-mentioned limits for the content of other nitrogen oxides in nitrous oxide apply not only to the hydroxylation of aromatics by means of nitrous oxide which has been obtained by reaction of ammonia and nitric acid but also to all other processes for the direct oxidation (hydroxylation) of aromatics, in particular of benzene to form phenol, by means of nitrous oxide. In particular, the limits also apply to nitrous oxide which has been prepared by other reactions, e.g. the reaction of ammonia with oxygen. If these reactions produce nitrous oxide whose content of other nitrogen oxides is above the preferred limits, the known methods of reducing the content of other nitrogen oxides can be used to reduce the content of nitrogen oxides to the level preferred according to the invention. Such a method is described, for example, in DE 100 09 639.

The hydroxylation is carried out in a manner known to those skilled in the art. Such processes are described, for example, in U.S. Pat. Nos. 5,001,280, 5,110,995 and U.S. Pat. No. 5,055,623. In these processes, a gaseous mixture of nitrous oxide with benzene and an inert gas is reacted in the presence of a zeolite catalyst at 400° C. The contact time in these processes is usually one second and the ratio of benzene to N₂ to N₂O is 2:5:8. Numerous changes to the parameters have been undertaken in various processes for the hydroxylation of aromatics which have at least one replaceable hydrogen atom on the aromatic ring, e.g. benzene. The ratio of nitrous oxide to inert gas to aromatic, in particular, has been varied over a wide range. The reaction temperatures and the catalysts used have likewise been varied.

In contrast to these processes, when the hydroxylation is carried out according to the invention under essentially identical process parameters, no inert gas, e.g. nitrogen, is mixed in. Instead, the aromatic to be hydroxylated is used as diluent. This does not interfere in the reaction and has to be removed as unreacted starting material from the product stream and recirculated in any case. Removing any inert gas used would represent an additional process step which can be avoided in the manner disclosed by the invention.

The aromatic used for the hydroxylation is preferably benzene or a benzene derivative, e.g. chlorobenzene, fluorobenzene, toluene, ethylbenzene or phenol.

The hydroxylation reaction is preferably carried out at a temperature above about 200° C., preferably a temperature of from about 200 to about 224° C., from about 225 to about 350° C., from about 351 to about 500° C., from about 501 to about 600° C. or from about 601 to about 850° C. The hydroxylation reaction is particularly preferably carried out at a temperature of from about 350 to about 500° C., very particularly preferably from about 400 to about 450° C.

The hydroxylation reaction is preferably carried out at a pressure of from 1 to 10 bar absolute, preferably a pressure of from about 1 to about 3.9 bar absolute, from about 4 to about 6.9 bar absolute or from about 7 to about 10 bar absolute. The hydroxylation reaction is particularly preferably carried out at a pressure of from about 2 to about 3.9 bar absolute.

In the process of the invention, the molar ratio of nitrous oxide or crude nitrous oxide from the reaction of ammonia with nitric acid to the aromatic to be hydroxylated, in particular benzene, in the hydroxylation is preferably from about 1:20 to about 1:2, more preferably from about 1:20 to about 1:15, from about 1:14 to about 1:10 or from about 1:9 to about 1:2. It has been found that the hydroxylation of aromatics by means of nitrous oxide can safely be carried out without use of an inert gas when the aromatic is used as diluent which can take up and remove the heat of reaction evolved. It should be noted that the aromatics frequently have a significantly greater molar heat capacity than nitrogen and are thus significantly more suitable for the removal of heat energy. Thus, for example, benzene has a molar heat capacity which is more than five times the heat capacity of nitrogen. The hydroxylation of aromatics by means of nitrous oxide can therefore also be carried out safely without the use of an inert gas when, for example, the abovementioned ratios of nitrous oxide to aromatic are employed.

As catalysts for the hydroxylation reaction, preference is given to using zeolites or silicalites, very particularly preferably iron-containing zeolites or silicalites. Very particular preference is given to using catalysts based on the zeolites ZSM-5 or ZSM-11 which contain from about 0.01 to about 5% by weight of iron. These catalysts are preferably used in the acid form.

The hydroxylation results in an offgas stream which comprises nitrogen formed in the reaction of nitrous oxide with the aromatic together with unreacted aromatic. The aromatics are recovered from the offgas stream, for example by condensation or absorption. The recovered aromatic can be returned to the hydroxylation. To enable the recovery of the aromatic from the offgas stream to be carried out economically, it is advantageous for as little nitrogen as possible to be introduced. The incoming nitrous oxide stream preferably contains not more than about 5% by weight of nitrogen, preferably not more than about 2% by weight of nitrogen.

The ammonia which is preferably used as starting material for the preparation of nitrous oxide in the process of the invention is industrially produced worldwide in large quantities and thus inexpensively, mostly by the Haber-Bosch process from nitrogen and hydrogen, and, as an important raw material for the chemical industry, is readily available.

The nitric acid can, for example, be prepared on an industrial scale by oxidation of ammonia to predominantly nitrogen monoxide in the Ostwald process. According to Ullmann's Encyclopedia of Industrial Chemistry, Volume A17, p. 293 ff., 1991, this oxidation of ammonia is one of the most effective catalytic reactions, in which yields of nitrogen monoxide of up to about 98%, based on ammonia, are achieved on an industrial scale. The second reactant is oxygen-containing gas, preferably air, or else pure oxygen or a mixture of the two, so that water is formed in addition to nitrogen monoxide. The proportion of ammonia in ammonia/air feed mixtures is generally determined by the lower explosive limit and is, depending the pressure conditions, up to about 13.5% by weight. This oxidation reaction is usually carried out at pressures up to 12 bar absolute and temperatures in the range from about 840° C. to about 950° C. Catalysts which have been found to be useful are, in particular, platinum gauzes which may contain a proportion of from 5 to 10% by weight of rhodium to improve the catalyst properties. Various types of reactor and reactor systems are known for carrying out the reaction, cf., for example, UlImann's Encyclopedia of Industrial Chemistry, Volume A17, p. 293 ff., 1991. For the ammonia oxidation, preference is given to using reactors having a heat recovery system for recovering the energy liberated in the reaction.

The main reactions occurring in the preparation of nitric acid are:

4NH₃+5O₂→4NO+6H₂O (II)

Oxidation of ammonia by means of oxygen to form nitrogen monoxide and water,

2NO+O₂→2NO₂ [symbol] N₂O₄ (III)

Oxidation of nitrogen monoxide to nitrogen dioxide or dinitrogen tetroxide and

3NO₂+H₂O→2HNO₃+NO (IV)

Absorption of nitrogen dioxide in water, which leads to the formation of nitric acid and nitrogen monoxide.

Nitrogen and nitrous oxide are formed to a small extent as byproducts under the abovementioned reaction conditions in this form of ammonia oxidation.

In cases in which the nitrous oxide prepared according to the invention is later used in processes for the hydroxylation of aromatics in which the content of nitrogen and/or water is critical, the product gas stream from the reaction of ammonia with nitric acid can firstly be subjected to one or more purification steps. If the water formed is to be separated off, the gas stream can be dried very simply in a manner known to those skilled in the art, for example by condensation or absorption of the water on molecular sieves or silica gels. When the nitrous oxide prepared according to the invention is to be used afterwards for the direct synthesis of phenol, such a drying step in the preparation of nitrous oxide is preferred in order to reliably rule out an influence on the hydroxylation of benzene. When the nitrous oxide prepared according to the invention is to be used later for direct phenol synthesis, preference is given to such a drying step in the nitrous oxide preparation, in order to completely rule out any influence on the hydroxylation of benzene. The content of the nitrogen formed as by-product can likewise be reduced, e.g. by rectification at high pressure, by liquefaction or by suitable membrane separation methods for separating off N₂.

In its combination of, in particular, the preparation of nitrous oxide from ammonia and nitric acid and the use of this nitrous oxide for the hydroxylation of aromatics, in particular the hydroxylation of benzene to phenol, the process of the invention thus has, owing to the ready availability of the starting materials at a favorable price, a high and economical potential for the preparation of hydroxylated aromatics, in particular phenol.

The process of the invention is illustrated by the following example, without being restricted thereto:

### EXAMPLE 1

300 g of ammonium nitrate, 300 ml of concentrated nitric acid, 300 ml of water and 15 ml of concentrated hydrochloric acid were mixed in an apparatus as described in U.S. Pat. No. 4,102,986, which comprises a reaction vessel, which contains the reaction mixture, three reservoirs, from which further quantities of a 60% strength ammonium nitrate solution, dilute (3M) hydrochloric acid and concentrated nitric acid can be metered into the reaction mixture, and a reflux condenser (column) at the top of the reaction vessel.

This reaction mixture was heated to a temperature corresponding to the boiling point of this mixture. The gases formed were passed into the reflux condenser where condensable gases, e.g. water vapor, were separated off from the other gases.

Further quantities of each of the starting materials were metered into the reaction mixture so that the concentration of hydrogen ions was from 5 to 5.5 M, that of chloride ions was less than 0.2 M and that of nitrate ions was from 12 to 14.5 M.

Nitrous oxide which had a concentration of other nitrogen oxides of 5 vppm and a nitrogen content of less than 10% by weight was obtained.

This nitrous oxide was used for the hydroxylation of benzene to phenol. The hydroxylation reaction was carried out in an adiabatic tube reactor. The tube reactor was charged with 500 g of an iron ZSM-5 zeolite having an iron content of 2% by weight. The reactor was operated at an inlet temperature of 400° C. and a pressure of 3.5 bar absolute.

2.5 kg/h of benzene (32 mol/h) and 0.1 kg/h of nitrous oxide (2.23 mol/h) were fed into the reactor. Condensation of the product stream obtained at the outlet from the reactor gave a liquid phase comprising benzene and phenol. This liquid phase was analyzed over the entire run. The nitrogen which was likewise present in the product stream was not condensed but was passed in gaseous form to offgas purification.

FIG. 1 shows the phenol concentration as a function of time from the start of the run in graph form. It can clearly be seen that the phenol concentration at the outlet from the reactor decreases with increasing reaction duration because of increasing blockage of the catalyst.

The Example described above is set forth solely to assist in the understanding of the invention. Thus, those skilled in the art will appreciate that the methods of the present invention can provide a method of peptide mapping of a polypeptide comprising one or more cysteine residues.

One skilled in the art would readily appreciate that the present invention is well adapted to carry out the objects and obtain the ends and advantages mentioned, as well as those inherent therein. The methods and procedures described herein are presently representative of preferred embodiments and are exemplary and are not intended as limitations on the scope of the invention. Changes therein and other uses will occur to those skilled in the art which are encompassed within the spirit of the invention.

It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions indicates the exclusion of equivalents of the features shown and described or portions thereof. It is recognized that various modifications are possible within the scope of the invention. Thus, it should be understood that although the present invention has been specifically disclosed by preferred embodiments and optional features, modification and variation of the concepts herein disclosed may be resorted to by those skilled in the art, and that such modifications and variations are considered to be falling within the scope of the invention, which is limited only by the following claims.

What is claimed is:

1. A process for the hydroxylation of aromatic hydrocarbons comprising adding nitrous oxide to an aromatic hydrocarbon in a hydroxylation reaction, wherein said nitrous oxide is obtained by reacting ammonia with nitric acid.

2. The process as claimed in claim 1, wherein the reaction of ammonia with nitric acid is carried out at a temperature of from about 80 to about 200° C.

3. The process as claimed in claim 1, wherein the reaction of ammonia with nitric acid is carried out in the presence of a catalyst comprising at least one metal selected from the group consisting of manganese, copper, cerium, lead, bismuth, cobalt and nickel.

4. The process as claimed in claim 1, wherein the nitrous oxide used for the hydroxylation has a nitrogen content of less than about 10% by weight.

5. The process as claimed in claim 1, wherein the nitrous oxide used for the hydroxylation has a content of other nitrogen oxides of less than 50 vppm.

6. The process as claimed in claim 1, wherein the hydroxylation is carried out at a temperature above about 200° C.

7. The process as claimed in claim 3, wherein zeolites or silicalites having an iron content of from about 0.01 to about 5% by weight are used as catalysts for the hydroxylation.

8. The process as claimed in claim 1, wherein the aromatic to be hydroxylated is at least one compound selected from among benzene, chlorobenzene, fluorobenzene, ethylbenzene and phenol.

9. The process as claimed in claim 1, wherein the molar ratio of aromatic to nitrous oxide in the hydroxylation is from about 2:1 to about 20:1.

10. A process for the hydroxylation of benzene to phenol comprising adding nitrous oxide to benzene in a hydroxylation reaction, wherein said nitrous oxide is obtained by reacting ammonia with nitric acid.

11. The process as claimed in claim 2, wherein the reaction of ammonia with nitric acid is carried out in the presence of a catalyst comprising at least one metal selected from the group consisting of manganese, copper, cerium, lead, bismuth, cobalt and nickel.

12. The process as claimed in claim 8, wherein the aromatic to be hydroxylated comprises mixtures of at least two compounds.
